(19) United States
(12) Patent Application Publication
(10) Pub. No.: US 2014/0295490 A1
(43) Pub. Date: Oct. 2, 2014

(54) FETAL RED BLOOD CELL DETECTION

(71) Applicant: Asta Fluidic Technologies, Inc., Goleta, CA (US)

(72) Inventors: Sumita Pennathur, Goleta, CA (US); Alejandro R. Soffici, Santa Barbara, CA (US); Alexander J. Russell, Goleta, CA (US); Anthony T. Chobot, Goleta, CA (US)

(73) Assignee: Asta Fluidic Technologies, Inc., Goleta, CA (US)

(21) Appl. No.: 14/169,043

(22) Filed: Jan. 30, 2014

Related U.S. Application Data
(60) Provisional application No. 61/758,472, filed on Jan. 30, 2013.

(51) Int. Cl. G01N 33/80

(52) U.S. Cl. CPC G01N 33/80
USPC 435/39; 435/287.3

(57) ABSTRACT

A device for analyzing a maternal blood sample for quantification of the percentage of fetal red blood cells present with respect to the number of maternal red blood cells includes reagents for mixing with the biological sample, a microfluidic chip, 5 fluid reservoirs, a pumping system, an image acquisition system, an image analysis system, and an electronic control board. The microfluidic channel can confine the objects of interest to a monolayer, and may trap them in an organized array for analysis. The device uses a reduced sample volume and microfluidic pumping and imaging techniques throughout. The disclosed invention holds distinct advantages over the current state of the art in fetal red blood cell quantification in a maternal blood sample by producing faster results, removing operator error, reducing 10 costs, and providing overall simplification of the testing and analysis procedure.

### CROSS-REFERENCE TO RELATED APPLICATIONS

This application claims the benefit under 35 U.S.C. §119(e) to U.S. Provisional App. No. 61/758,472 entitled FETAL RED BLOOD DETECTION, which is hereby incorporated by reference in its entirety.

### TECHNICAL FIELD

This invention relates broadly to devices and methods of human red blood cell (RBC) analysis.

### BACKGROUND OF THE INVENTION

Bleeding across the placenta, from the fetus to the mother, sometimes occurs during pregnancy. This bleeding occurs to some degree in all pregnancies, but can be accelerated by preexisting conditions or by a trauma incident to the mother. In the case of a pregnancy where the mother is blood type Rhesus D-negative and the fetus is Rhesus D-positive, the mother may begin to develop antibodies that reject the current, or future, fetuses. Because fetal blood testing during pregnancy is an invasive and potentially harmful procedure, sometimes mothers are treated at the first indication of bleeding in lieu of fetal blood type determination. Additional treatment is administered to the mother post-birth in the case of an identified blood type mismatch, as previously described. The treatment, Rh Immune Globulin (RhIG), is administered by doctors to at risk mothers in doses proportional to the percent RBCs in the mother's circulation to prevent the development of such antibodies. Additional sampling of fetal RBCs is performed to quantify excessive bleeding in the case of severe fetomaternal hemorrhage. Further screening for fetomaternal hemorrhage may be relevant in all pregnancies if a device can be made to perform quantification of fetal RBCs in circulation in a rapid and cost effective manner. This treatment is advantageous because it does not require direct sampling of the fetus, but rather quantification of the number of fetal RBCs in a sample of the mother's blood. One method to quantify the percentage of fetal RBCs in a pregnant woman's circulation is with the Kleihauer-Betke (KB) acid elution test, wherein a blood sample is processed and analyzed by a technician. This method is time consuming, cumbersome and prone to human error. An alternative method of detection and quantification is by use of Flow Cytometry devices, wherein the cells of interest are chemically or biologically tagged and imaged by a machine. This machine is often reserved for more complex tests for which there is no alternative method of detection. Flow Cytometry devices are additionally expensive to operate. It can be therefore beneficial to develop an automated test to quantify the percent fetal RBCs in a pregnant women's circulation.

The field of microfluidic technology has been developed through the coupling of micro-electro-mechanical-systems (MEMS) fabrication techniques, which were initially developed in the semiconductor industry, to fluid systems. One application of microfluidic devices is in the field of biological sample detection. An automated test using relevant microfluidic techniques to quantify the percent fetal RBCs in a pregnant women's circulation is needed.

### SUMMARY OF THE INVENTION

According to an aspect of the invention, a device includes reagents for mixing with a sample. In one embodiment, the reagents include an acidic buffer solution, and a phosphate buffered saline solution. The reagents and sample are mixed prior to or after insertion into the device.

According to another aspect of the invention, a device includes a microfluidic chip for viewing the objects of interest, containing reagents, and mixing the reagents and sample. The microfluidic chip directs flow through a microfluidic channel. The microfluidic chip can have dedicated fluid mixing zones, and object of interest imaging and trapping zones. The microfluidic chip has fluid inlets and outlets for interfacing the microfluidic channel with the surrounding environment and external fluids. The microfluidic channel confines the objects of interest to a monolayer through geometric constraints to prevent clogging and facilitate imaging. The microfluidic chip is additionally wholly or partially optically transparent to facilitate imaging. There may be convergence of microfluidic channels to interface and combine multiple fluid inlets.

According to another aspect of the invention, a device includes fluid reservoirs for interfacing, housing and mixing reagents and samples. In one embodiment the fluid reservoirs are located within the device, external to the microfluidic chip. In this embodiment fluids are added to the fluid reservoirs prior to running the device. In another embodiment, one or more of the fluid reservoirs are located on the microfluidic chip. In this embodiment the fluid reservoirs are filled during the manufacturing and packaging of the microfluidic chip and are interfaced with the sample in the fluid mixing zone for object of interest imaging through converged microfluidic channels.

According to another aspect of the invention, a device includes a pumping system to facilitate fluid flow throughout the device. In one embodiment the pumping system is located between the fluid reservoirs and the microfluidic chip. It is connected with separate or combined fluid inlet and outlet conduits. The fluid pumping system can be active or passive.

According to another aspect of the invention, a device includes an image acquisition system for capturing images of the objects of interest. In one embodiment the image acquisition system comprises of a light source and a light detector. The light source illuminates the microfluidic chip, channel, and objects of interest for imaging by the light detector. In one embodiment, the light source is an LED. The light detector is a CCD in one embodiment, and a CMOS in another embodiment. The field of view of the light detector can either cover the entire imaging area, or the light detector can be mounted to a translational stage for complete coverage of the imaging area by the field of view of the light detector.

According to another aspect of the invention, a device includes an image analysis system comprising of an image analysis algorithm. The image analysis algorithm uses the differences in captured light intensity, the coordinates at each pixel, and the coordinates of the translatable stage for determining the location and intensity of the objects of interest. In one embodiment, the image analysis algorithm comprises an edge interpolation method to distinguish the boundary of objects of interest. Using differences in intensity between objects of interest, the image analysis algorithm differentiates between the species present.

According to another aspect of the invention, a device includes an electronic control board that is used to control and process a set of sensors and actuators comprising: a pumping system; an image acquisition system; and an image analysis system. In one embodiment, the electronic control board is a microcontroller. In one embodiment, the electronic control board actuates the image capturing device at predetermined time intervals. In another embodiment, the electronic control board positions the image acquisition system using the translation stage and actuates the image capturing device at predetermined time intervals. The electronic control board can additionally be used to actuate the pumping system. In one embodiment, the electronic control board processes the image analysis algorithm in addition to quantifying the percentage of fetal blood cells present in a maternal blood sample.

Aspects of the invention can be advantageous compared to the current state of the art in fetal RBC quantification in a maternal blood sample by producing faster results, removing operating error, reducing costs, and providing overall simplification of the testing and analysis procedure.

### BRIEF DESCRIPTION OF THE DRAWINGS

FIG. 1 is a schematic of one embodiment of the device showing fluid reservoirs, a microfluidic chip, pumps, fluid conduits, and an imaging system.

FIG. 2 is a schematic of one embodiment of the imaging system showing an image capturing device and a translational stage.

FIG. 3 is a schematic of a top down view of one embodiment of a microfluidic chip showing one fluid inlet, microfluidic channels, an on-chip reservoir, a dedicated mixing space, and one fluid outlet.

FIG. 4 is a schematic of a top down view of a second embodiment of a microfluidic chip showing two fluid inlets, microfluidic channels, microfluidic channel convergence, a dedicated mixing space, additional mixing space in a serpentine microfluidic channel, and one fluid outlet. This embodiment might be used in a device where the imaging system does not move.

FIG. 5 is a schematic of a top down view of a third embodiment of a microfluidic chip showing one fluid inlet, a zone for capturing objects of interest, and one fluid outlet.

FIG. 6 is a schematic of a side view of one embodiment of a microfluidic chip showing one fluid conduit entering one fluid inlet, a microfluidic channel, one fluid conduit exiting one fluid outlet, and a side view of one embodiment of an image capturing device.

### DETAILED DESCRIPTION

The invention provides, in some aspects, methods and a device, either portable or of stationary form, to efficiently and accurately determine the percentage of fetal red blood cells (RBCs) compared to maternal RBCs in a blood sample of a woman, during or after pregnancy, or in control blood samples containing known amounts of fetal RBCs compared to adult RBCs. Among others, one type of blood sample is a maternal cord blood sample. In one embodiment, the blood sample is diluted with a Phosphate Buffered Saline solution. In another embodiment, the blood sample is diluted by greater than 50% by the Phosphate Buffered Saline solution. In yet another embodiment, the blood sample is diluted by greater than 90% by the Phosphate Buffered Saline (PBS) solution. The degree of dilution can be relevant with regard to reagent consumption during the detection process in addition to altering the viscosity of the blood sample; increased dilution lowers the viscosity by reducing the number of RBCs per unit volume. The exact dilution in some cases is important in understanding the RBC concentration for imaging purposes.

According to an aspect of the invention, the device includes reagents for mixing with the blood sample, a microfluidic chip, fluid reservoirs, a pumping system, an image acquisition system, an image analysis algorithm, and an electronic control board.

Microfluidic devices can create a single, non-overlapping layer of fRBCs and RBCs using CAD software and rapid prototyping equipment. A single layer of RBCs can be prepared using channel height confinement. A single layer of RBCs can be achieved with this method by fabricating channels with a height equal to 90% of the thickness of a RBC. Upon capillary filling of the microfluidic channel with a blood sample, the constriction causes the fRBCs and RBCs to deform and align to a consistent orientation upon entering the channel. The constriction further prevents fRBCs and RBCs from overlapping, which can be important for differentiation in some cases.

In addition to confinement of RBCs to a single layer by channel height modulation, novel cell and particle ordering designs can be utilized. Hydrodynamic focusing and/or inertial effects (such as dean vortices) can create a single stream of particles at a very rapid (>3,000 cells/second) rate. Individual cells can be held in a microarray of cell traps for identification/differentiation.

Microfluidic geometries can be fabricated using a hot embossing technique optimized for plastic microfluidic chip fabrication. This rapid, low cost fabrication method can be enabled by an innovative nickel mold fabrication process that can turn a CAD model into a batch of plastic microfluidic chips quickly and inexpensively.

Systems and methods can include a microfluidic Tee based chip, which can receive a blood sample in one inlet and a citric acid buffer in the other. Syringe pumps can be used to pump the fluids through the microfluidic chip and to generate a homogenous mixture. The mixing region can be characterized according to optical interrogation of the sample composition across the channel cross section. Additional mixing stimulus such as pillars, S-curves, and barriers to flow can also be used. The mixing ratio and mixing time of the sample and acidic buffer can be optimized to achieve satisfactory fRBC and RBC differentiation on chip. The differential elution of fetal and maternal red blood cells is known to be time dependent. Supplemental on chip staining processes can be considered, as well as altogether different methods such as those described herein, where Dean vortices are used place cells in a specific place in the channel based on size.

Optical differentiation can be performed using a portable platform. Fluid interface components can be developed to interface a microfluidic chip with a portable detection platform. Certain fluid interface components can allow for ‘microfluidic breadboarding’ and include syringe pumps, automated valves, valve manifolds and computer interface controllers, capillary tubing, chip port connectors, and controller automation computer software. The components can be networked to the microfluidic chip to mix a blood sample with acidic buffer at a defined ratio for a defined amount of time, and then prepare a single, non-overlapping layer of fRBCs and RBCs. The blood sample and acidic buffer can be stored in syringe pumps, pumped to the microfluidic chip through two access ports, and subsequently mixed on chip.

According to an aspect of the invention, reagents are used to create a detectable difference between fetal and maternal RBCs. In one embodiment the reagents are used to optically differentiate the fetal and maternal RBCs. The RBC differentiation procedure can be established through a differential resistance to an acidic environment that is exhibited by maternal and fetal RBCs. In this embodiment, the fetal RBCs are more resistant to the acidic environment, whereas the maternal RBCs release their hemoglobin in a process known as hemolysis. The acidic environment used to differentiate the cells in the aforementioned embodiments can be aqueous. In another embodiment, the RBCs are further differentiated through a staining process in which the fetal and maternal RBCs experience a differential affinity to a staining solution, which can be aqueous.

In one embodiment, the acidic solution is a solution of pH between 2.6 and 7. In one embodiment, the acidic solution is a Citrate Phosphate Buffer. In a refinement to this embodiment, the Citrate Phosphate Buffer has a pH between 4 and 6.

In one embodiment, the staining solution is a solution which stains the fetal and maternal cells with different colors or intensities. In one embodiment, the staining solution is Erythrosin-B or similar.

According to an aspect of the invention, the creation of a detectable difference between fetal and maternal RBCs is performed prior to imaging the blood samples. In one embodiment, the differentiation procedure, wherein reagents are mixed with the blood sample, is performed prior to inserting the sample into the device.

In another embodiment, the differentiation procedure is performed within the device. There are multiple embodiments wherein the device obtains the necessary fluids for, and performs, the mixing. In one embodiment, the fluids are inserted into the device by the user. In one embodiment, the fluids are mixed by the device in a fluid reservoir, prior to the microfluidic chip. In one embodiment, the fluids are mixed in the microfluidic chip. In one embodiment, the reagents are housed within the microfluidic chip and mixing with the sample is performed on the microfluidic chip. In one embodiment, the mixing procedure is passive in that it does not require a power source. In one embodiment, the mixing procedure is performed by an actuator that requires an electrical power source.

According to an aspect of the invention, fluid reservoirs are used to contain at least one of reagents and blood samples within the device. Fluid reservoirs interface external fluids with the device. In one embodiment, a fluid reservoir will be used to contain the prepared sample and reagent mixture, diluted with PBS or otherwise. In another embodiment, multiple fluid reservoirs will be used to contain individually, or in any combination thereof, a maternal blood sample, an acidic solution, a staining solution, a PBS solution, and a deionized water solution. In one embodiment, an additional reservoir contains one or more solutions for cleaning and flushing the plumbing system that exists within, upstream, and downstream of the fluid reservoirs.

In one embodiment, the fluid reservoirs are disposable. In another embodiment, the fluid reservoirs are permanently fixed within the device. In yet another embodiment, the fluid reservoirs are fixed within the device but can be any or all of removed, cleaned and replaced. In one embodiment, the fluid reservoirs contain sufficient fluid for one device run. In one embodiment, the fluid reservoirs contain sufficient fluid for multiple device runs.

In one embodiment, the fluid reservoirs are sealed from the surrounding environment. In one embodiment, the fluid reservoirs are filled and sealed to the device through the same interface. In one embodiment, the fluid reservoirs are filled and sealed to the device through different interfaces. In one embodiment, sealing from the environment is performed by a lid. In one embodiment, the lid is removable, hinged, or deformable. In one embodiment, the lid snaps into place. In one embodiment, the lid is screwed into place. In one embodiment, the lid, a portion, or the whole reservoir, is fabricated of a material that can be penetrated by a needle. In one embodiment, the material that is penetrable by needle is used for one or both of filling the reservoir and interfacing the reservoir to the fluid handling portion of the device.

In one embodiment, one or more of the fluid reservoirs are interfaced to the microfluidic chip by tubes or pipes. In one embodiment, the inner diameter of the tube or pipe is less than 500 μm. In one embodiment, the inner diameter of the tubing is less than 100 μm. In one embodiment, the inner diameter of the tubing is altered to promote capillary filling. In another embodiment, the inner diameter of the tubing is altered to control one or more volume flow rates.

In one embodiment, one or more of the fluid reservoirs are located directly in contact with the entrance to the microfluidic chip. In one embodiment, one or more of the fluid reservoirs are attached to the microfluidic chip. In one embodiment, one or more of the fluid reservoirs are packaged within the microfluidic chip. In one embodiment, one or more fluid reservoirs are interfaced to one or more additional reservoirs for mixing prior to interfacing with the microfluidic chip.

According to an aspect of the invention, fluids are transported from the reservoirs to one or more imaging areas on the microfluidic chip by a potential flow. In one embodiment, the potential flow is generated by a pump. In one embodiment, the pump causes fluid locomotion through one or more of the following mechanisms: gravity, electroosmosis, capillary forces, peristaltic pumping, pressure volume work, or vacuum. In one embodiment, pressure volume work is performed by a pressurized canister. In one embodiment, pressure volume work is performed by an attached pressurized tubing or hose. In one embodiment, a vacuum is created within the device using a piston or pump. In one embodiment, the vacuum is created during the manufacturing and packaging of the microfluidic chip. In one embodiment, one or more of the pumping mechanisms is passive. In one embodiment, one or more of the pumping mechanisms is active.

According to an aspect of the invention, a microfluidic chip is used to interface one or more fluids from the fluid reservoirs to the imaging area. In one embodiment, the imaging area is located within the microfluidic chip. In one embodiment, there is one fluid inlet on the microfluidic chip for every fluid that is pumped to the imaging area from outside of the microfluidic chip. In one embodiment, the fluid inlets connect to one or more fluid channels in the microfluidic chip. In one embodiment, there are existing reservoirs containing reagents on the chip. In one embodiment, the on-chip fluid reservoirs are connected to fluid channels within the chip. In one embodiment, the on-chip fluid reservoirs are actuated to allow flow by an external stimulus. In one embodiment, the on-chip fluid reservoirs are passively opened to allow flow. In one embodiment, fluid channels converge to promote mixing of the sample and reagents within the chip. In one embodiment, there is a discrete fluid mixing reservoir or zone on the chip. In one embodiment, fluid mixing occurs within the converged channels. In one embodiment, there are one or more fluid outlets for either relieving internal pressures or to release fluids.

In one embodiment the microfluidic chip is used to confine the RBCs to a monolayer, a single layer of cells. In one embodiment, a monolayer is achieved by geometric constraints of the fluid channels in the microfluidic chip. In one embodiment, the fluid channel in the microfluidic chip is fewer than 10 μm in height. In one embodiment, the fluid channel in the microfluidic chip is greater than 5 μm in height. The height of the channel for which the RBCs remain in a monolayer is a function of the pressure generated by the pumping system and the dilution value of the blood sample with PBS. In yet another embodiment the microfluidic chip does not confine the RBCs to a monolayer, or only a portion of the chip confines RBCs to a single layer.

In one embodiment, the microfluidic chip is optically transparent. In one embodiment, the entire chip is optically transparent. In one embodiment, portions of the chip are optically transparent. In one embodiment, one or more imaging areas are optically transparent. It is important in some cases that portions of the microfluidic chip are transparent for imaging and inspection purposes.

In one embodiment, fluids are continually passed through an imaging area for detection by the imaging system. In one embodiment, there are locations on the microfluidic chip where objects of interest are trapped while the remaining fluids are passed. In one embodiment, the objects of interest are trapped by a geometric constraint within the channel. In one embodiment, objects of interest are imaged while they are flowing within the channel. In one embodiment, objects of interest are imaged while they are trapped within the channel. In one embodiment, the objects of interest are fetal and maternal RBCs. In one embodiment, there is one imaging area on the microfluidic chip. In one embodiment, there are multiple imaging locations on the microfluidic chip. In one embodiment, there are multiple, discrete, imaging locations on the microfluidic chip.

According to an aspect of the invention, there is an image acquisition system for capturing images of the objects of interest within the microfluidic chip, comprising of a light source and detector. In one embodiment, the image acquisition system is in contact with the microfluidic chip. In one embodiment, the image acquisition system is located adjacent to the microfluidic chip at a distance that is roughly equal to the focal plane of the image acquisition system. In one embodiment, the field of view of the image acquisition system covers the entire area of interest on the microfluidic chip. In one embodiment, the image acquisition system is stationary. In one embodiment, the image acquisition system can be translated in space to cover the entire area of interest, or a portion thereof, with the field of view of the imaging system.

In one embodiment, a light source is used for illumination of the area of interest. In one embodiment, the light source used for illuminating the area of interest is a light-emitting-diode (LED). In one embodiment, images are captured through the use of a charge-coupled-device (CCD). In one embodiment, images are captured through the use of a complementary-metal-oxide-semiconductor (CMOS). In one embodiment, the CCD or CMOS is directly imaging the microfluidic chip. In one embodiment, objective lenses are used to magnify the area of interest on the microfluidic chip. In one embodiment, image detection and illumination are on opposing sides of the microfluidic chip. In one embodiment, image detection is performed from the top or bottom sides of the microfluidic chip. In one embodiment, the area of interest is illuminated by a light source oriented 90 degrees from the image detector.

According to an aspect of the invention, there is an image analysis algorithm for analyzing the images that are captured by the image acquisition system. In one embodiment, the image analysis algorithm uses differences in light intensity at each pixel during the image analysis. In one embodiment, the image analysis algorithm will use an edge aware interpolation to distinguish individual cells. In one embodiment, the image analysis algorithm will count both the maternal and fetal RBCs. In one embodiment, the image analysis algorithm will use these counts to determine the percentage of fetal cells in comparison to the total number of cells in circulation.

According to an aspect of the invention, there is an electronic control board to control pumps, image acquisition, and image analysis. In one embodiment, the electronic control board is a commercially available or substantially similar microcontroller, such as an Arduino or Raspberry Pi. In another embodiment of the invention, the electronic control board is, or consists of, a field programmable gate array (FPGA). In another embodiment of the invention, the electronic control board is a computer or portions thereof. In one embodiment, the electronic control board will actuate the pumps within the device. In one embodiment, the electronic control board will actuate the image acquisition hardware. In one embodiment, the electronic control board will process the images using the image analysis algorithm. In one embodiment, there is an external display screen to display results and commands to the user. In one embodiment, there is an external display screen that is controlled by the electronic control board. In another embodiment the display screen features a human touch interface.

According to an aspect of the invention wherein the total time to detect or quantify fetal RBCs or to detect or quantify FMH occurs in fewer than 30 minutes, or fewer than 15 minutes, or fewer than 10 minutes, or fewer than 5 minutes, or fewer than one minute. According to an aspect of the invention, fetal RBCs are detected or quantified using micron scale fluid pathways microfluidic technology.

There are several methods and materials for fabricating the microfluidic device, or fluid pathways, in which the sample containing fetal and maternal RBCs passes. According to one method of fabricating the microfluidic device wherein the microfluidic device is made of one of the following materials or classes of materials or similar material: polymers, plastics, thermoplastic, Poly(methyl methacrylate) (PMMA), Polycarbonate (PC), Polysulfone (PS), Polydimethylsiloxane (PDMS), Silicon dioxide, Fused silica, Amorphous silica, Quartz, Glass, Quartz glass, Silicon, Silicon derivative, Topas brand medical grade polymers, or Zeonex brand medical grade thermoplastics. According to one method of fabricating the microfluidic device wherein the material is fabricated according to one of the following procedures or methods: molding, injection molding, casting, injection casting, CNC machining, CNC micromachining, photolithography based micromachining, or any similar MEMS fabrication technique.

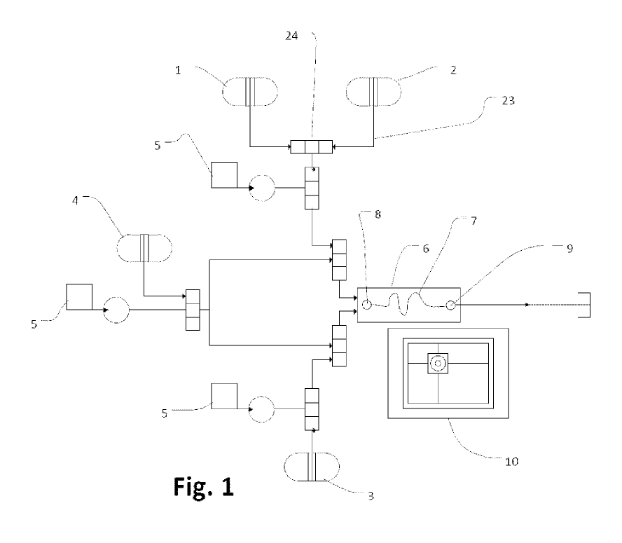

FIG. 1 is a schematic of one embodiment of the device showing fluid reservoirs, a microfluidic chip, pumps, fluid conduits, and an imaging system. The schematic depicts a sample reservoir, 1, and a PBS reservoir, 2. In this schematic, these two reservoirs are connected by fluid conduits, 23, to a three way valve, 24. The three way valve is thereby connected to a second three way valve and a syringe pump, 5, and another fluid conduit. The syringe pump selectively draws fluids out of reservoirs 1 and 2 and into the syringe before expelling the fluid through the attached fluid conduit. The fluids then travel to another three way valve via a fluid conduit which directs flow to the inlet, 8, of the microfluidic chip, 6. The sample, diluted with PBS, is mixed with a buffer from reservoir 3 that is pumped by syringe pump 5 to the inlet of the microfluidic chip in a similar fashion to the sample. The sample and buffer travel through the microfluidic channel, 7, towards the fluid outlet, 9, of the microfluidic chip. While the solution travels through the microfluidic channel, the sample is imaged with the image acquisition system, 10. A reservoir of cleaning solution, 4, can be pumped through all of the fluid conduit system and microfluidic channel using clever actuation of the three way valves. The cleaning procedure prepares the device for use with a subsequent sample.

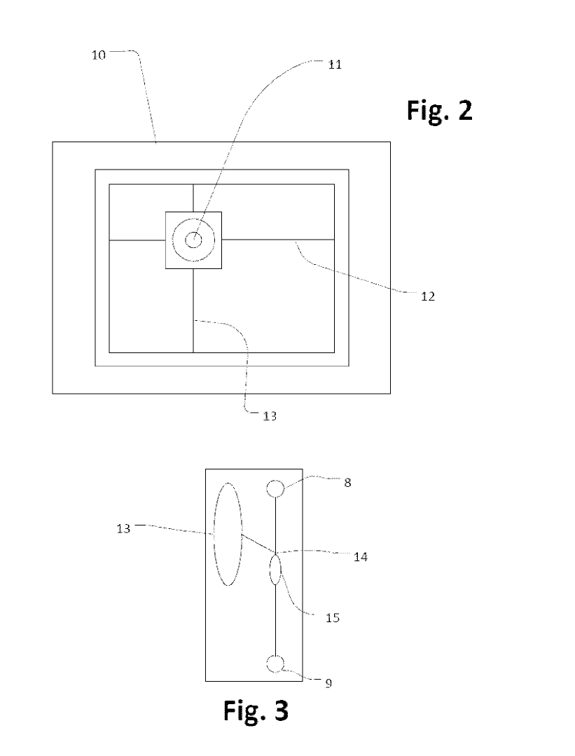

FIG. 2 is a schematic of one embodiment of the imaging system showing an image capturing device and a translational stage. The imaging system, 10, contains an image capturing device, 11, mounted to two orthogonal worm gears, 12 and 13, for complete coverage of the area of interest of the microfluidic chip.

FIG. 3 is a schematic of a top down view of one embodiment of a microfluidic chip showing one fluid inlet, microfluidic channels, an on-chip reservoir, a dedicated mixing space, and one fluid outlet. The inlet, 8, to the microfluidic chip and channel, converges, 14, with a microfluidic channel connected to an on-chip reservoir, 13, that is pre-filled during the microfluidic chip fabrication and packaging process. After convergence, the fluids mix in the dedicated on chip mixing zone, 15, before being imaged in the downstream microfluidic channel and exiting through the fluid outlet, 9.

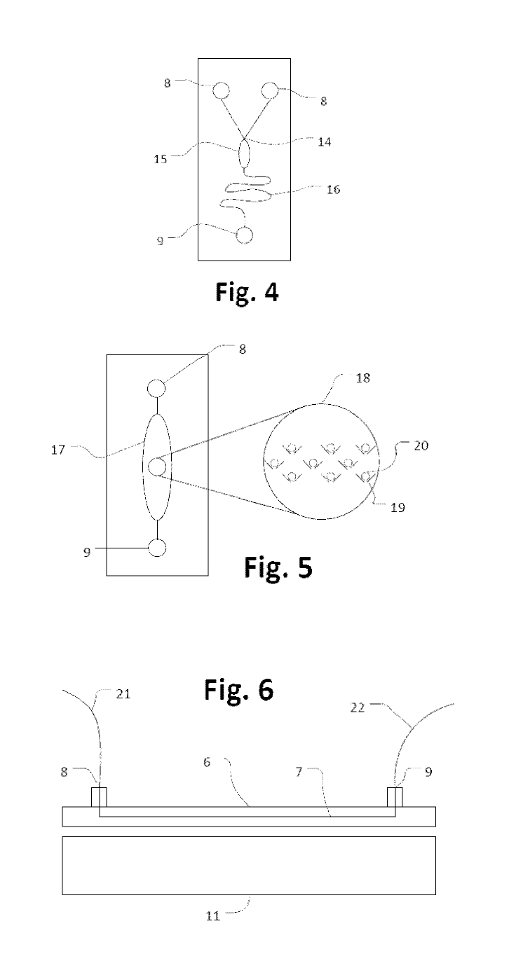

FIG. 4 is a schematic of a top down view of a second embodiment of a microfluidic chip showing two fluid inlets, microfluidic channels, microfluidic channel convergence, a dedicated mixing space, additional mixing space in a serpentine microfluidic channel, and one fluid outlet. This embodiment might be used in a device where the imaging system does not move. The two fluid inlets, 8, converge, 14, and mix in the dedicated mixing zone, 15, before travelling through a serpentine microfluidic channel, 16, wherein further mixing and imaging occurs before the fluid is expelled through the fluid outlet, 9.

FIG. 5 is a schematic of a top down view of a third embodiment of a microfluidic chip showing one fluid inlet, a zone for trapping and imaging objects of interest, and one fluid outlet. Fluid enters the microfluidic chip and channel through the fluid inlet, 8, wherein the channel enters an object of interest trapping and imaging zone,

17. A zoomed schematic of the trapping and imaging zone, 18, shows an array of traps, 19, which in this embodiment consist of two sloped extrusions. A trapped object of interest is show as 20. The fluid passes through the trapping and imaging zone and exits through the fluid outlet, 9.

FIG. 6 is a schematic of a side view of one embodiment of a microfluidic chip showing one fluid conduit entering one fluid inlet, a microfluidic channel, one fluid conduit exiting one fluid outlet, and a side view of one embodiment of an image capturing device. Fluid is pumped to the microfluidic chip via an upstream fluid conduit, 21, where it enters the microfluidic chip, 6, through a fluid inlet, 8, into a microfluidic channel, 7, whereupon it is imaged by the image capturing device, 11, and expelled through the fluid outlet, 9, and into the downstream fluid conduit, 22.

Although the invention has been shown and described with respect to a certain preferred embodiment or embodiments, it is obvious that equivalent alterations and modifications will occur to others skilled in the art upon reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1-20. (canceled)

21. A system for screening red blood cells, comprising:
a microfluidic device comprising a first inlet, an outlet, and a microfluidic flow channel fluidly connected to the first inlet, the first inlet configured to allow flow of a sample comprising red blood cells therethrough, the channel configured to have a geometry such that red blood cells flowing through the channel form a monolayer within the channel;
an image sensor configured to image at least part of the monolayer of red blood cells within the channel; and
an image analysis system configured to differentiate a first species of red blood cells from a second species of red blood cells, the image processor further configured to quantify the ratio of the first species of red blood cells to the second species of red blood cells using an image analysis algorithm.

22. The system of claim 21, wherein the image analysis system is configured to differentiate between fetal red blood cells and maternal red blood cells, and quantify the ratio of fetal red blood cells to maternal blood cells using the image analysis algorithm.

23. The system of claim 21, wherein the channel has a height equal to about 90% of the thickness of an RBC.

24. The system of claim 21, wherein the channel has a height of less than about 10 micrometers.

25. The system of claim 21, wherein the channel has a height of less than about 5 micrometers.

26. The system of claim 21, wherein the channel is optically transparent.

27. The system of claim 21, further comprising an acid buffer reagent.

28. The system of claim 21, further comprising a staining reagent.

29. The system of claim 21, further comprising at least one reservoir operably connected to the microfluidic device, the at least one reservoir operably connected with the microfluidic flow channel.

30. The system of claim 21, further comprising a fluidic mixing zone downstream of the first inlet and fluidly connected to the channel.

31. A method of screening for fetomaternal hemorrhage, comprising the steps of:
flowing red blood cells into an optically-transparent channel of a microfluidic device such that the red blood cells form a monolayer within the channel by virtue of geometric constraints of the channel, the channel having a height of less than 10 micrometers; and
analyzing the red blood cells within the channel, wherein analyzing the red blood cells comprises imaging the red blood cells, differentiating fetal red blood cells from maternal red blood cells based upon the imaging, and determining the ratio of fetal red blood cells to maternal red blood cells using a computer-based algorithm.

32. The method of claim 31, further comprising differentially eluting the red blood cells, such that the time to elute maternal red blood cells with respect to fetal red blood cells is optimized to differentiate fetal red blood cells from maternal red blood cells.

33. The method of claim 31, further comprising combining a blood sample comprising red blood cells with an acid buffer reagent such that the red blood cells are acid treated.

34. The method of claim 33, wherein the combining step occurs on the microfluidic device.

35. The method of claim 31, further comprising staining the red blood cells with a staining reagent.

36. The method of claim 31 further comprising flushing the channel with a cleaning solution after the flowing step.

37. The method of claim 31, wherein the method is performed in less than 15 minutes.

38. The method of claim 31, wherein the channel has a height equal to about 90% of the thickness of an RBC.

39. A method of creating a red blood cell monolayer, comprising:
flowing a blood sample containing red blood cells through a flow channel on a microfluidic device, the flow channel geometrically configured to cause the red blood cells to form a monolayer, the flow channel having a height of less than 10 micrometers.

40. The method of claim 39, further comprising analyzing the red blood cells using an image analysis system, wherein analyzing the red blood cells comprises differentiating a first species of red blood cells from a second species of red blood cells, and quantifying the ratio of the first species of red blood cells to the second species of red blood cells using a computer-based algorithm.
